(19) United States
(12) Patent Application Publication
(10) Pub. No.: US 2026/0120881 A1
(43) Pub. Date: Apr. 30, 2026

(54) METHOD FOR CONSTRUCTING COMORBIDITY PREDICTION MODEL OF DISEASES

(71) Applicants: National Central University, Taoyuan City (TW); Landseed International Hospital, Taoyuan City (TW); Phalanx Biotech Group, Inc., JUBEI CITY (TW)

(72) Inventors: Li-Jen Su, New Taipei City (TW); Jing-Hong Xiao, Taoyuan City (TW); Li-Ching Wu, Taipei City (TW); Hsiao-Yen Kang, Taoyuan City (TW); Tien Hsu, Taichung City (TW); Chin-Pyng Wu, Taipei City (TW)

(21) Appl. No.: 18/999,906

(22) Filed: Dec. 23, 2024

(30) Foreign Application Priority Data
Oct. 29, 2024 (TW) 113141321

(51) Int. Cl. G16H 50/30

(52) U.S. Cl. CPC G16H 50/30

(57) ABSTRACT

A method for constructing a comorbidity prediction model is provided. The method includes receiving a sample dataset, filtering and analyzing the dataset, and using harmonic centrality and betweenness centrality to identify critical core diseases and bridge diseases, thereby establishing a comorbidity prediction model.

### CROSS-REFERENCE TO RELATED APPLICATION

This application claims the priority benefit of Taiwan application serial no. 113141321, filed on Oct. 29, 2024, the full disclosure of which is incorporated herein by reference.

### BACKGROUND

### Technical Field

The present invention relates to a method for constructing a prediction model, and more particularly, to a method for constructing a comorbidity prediction model of diseases.

### Description of Related Art

In the medical field, comorbidity refers to the presence of one or more additional diseases that co-occur with a primary disease. According to statistics from Taiwan's Ministry of Health and Welfare, over 60% of seniors aged 65 and above in Taiwan have hypertension, nearly 30% have diabetes, 40% have hyperlipidemia, and close to 90% have at least one chronic disease. Additionally, more than half of the elderly population has three or more chronic conditions. Compared to individuals with a single disease, those with multiple chronic conditions generally experience a lower quality of life, as each chronic disease may negatively impact their well-being. Comorbidity also complicates medical decision-making, as patients often consult multiple specialists, leading to an increased likelihood of polypharmacy, drug interactions, and a higher risk of adverse reactions.

Accordingly, how to design a comorbidity prediction method capable of predicting the likelihood of future occurrence of related diseases is important.

### SUMMARY

In one aspect, the present invention provides a method for constructing a comorbidity prediction model of diseases. The method comprises (1) receiving a sample dataset that comprises a plurality of disease names or codes for different diseases and a plurality of patient counts for each respective disease; (2) conducting a pairwise chi-square test on each individual disease in the sample data to determine an association between each pair of diseases, and retaining disease relationships with a p-value less than a specified threshold; and (3) identifying at least one key core disease and at least one bridge disease by calculating harmonic centrality and betweenness centrality to establish the comorbidity prediction model.

According to an embodiment of this invention, the method further comprises excluding data from the sample dataset based on a predetermined threshold, wherein the predetermined threshold comprises diseases with fewer than two consultations within a one-year period before performing step (1).

According to an embodiment of this invention, the method further comprises categorizing the patient counts in the sample dataset by quartiles and retaining diseases with patient counts in the top 50% or 75% after performing step (2).

According to an embodiment of this invention, the method further comprises calculating a lift between each pair of the diseases in the sample dataset using an association rule and selecting; and retaining diseases with lift values in the top 25% after performing step (2).

According to an embodiment of this invention, the specified threshold is 0.05.

According to an embodiment of this invention, the harmonic centrality is calculated by a formula of

$$H ( s ) = \frac{1}{n - 1} \cdot {\sum }_{s \neq t} \frac{1}{d ( s , t )} ,$$

where H(s) represents the harmonic centrality score; n is the number of disease nodes; s and t denote distinct disease nodes; and d(s,t) is the shortest path length from s to t.

The betweenness centrality is calculated by a formula of

$${C}_{B} ( v ) = {\sum }_{s \neq v \neq t} \frac{{σ}_{s t} ( v )}{{σ}_{s t}} ,$$

where CB(v) represents the betweenness centrality score; s, t, and v denote distinct disease nodes; σₛₜ(v) is a number of shortest paths from s to t that pass-through v; and σₛₜ is a total number of shortest paths from s to t.

According to an embodiment of this invention, nodes with harmonic centrality scores in the top 25% are retained.

According to an embodiment of this invention, the threshold for the betweenness centrality is greater than 1.5 times the interquartile range (IQR).

According to an embodiment of this invention, the method further comprises calculating the network average path length of the sample dataset to construct a multi-layer network structure by a formula of

$${l}_{g} = \frac{1}{E \cdot ( E - 1 )} \cdot {\sum }_{s \neq t} d ( s , t ) ;$$

where lg represents the network average path length; E is the number of connections between any two of the disease nodes; s and t denote distinct disease nodes; and d(s,t) is the shortest path length from s to t. The network average path length determines the number of layers in the multi-layer network structure.

The above summary is intended to provide a simplified overview of the present invention to give the reader a basic understanding of its content. This summary is not a complete description of the invention and is not intended to highlight essential or critical elements of the embodiments or define the scope of the invention. After reviewing the following embodiments, those skilled in the relevant field will readily understand the fundamental spirit, additional aspects, technical means, and implementations of the present invention.

### BRIEF DESCRIPTION OF THE DRAWINGS

To make the above and other objectives, features, advantages, and embodiments of the present invention more comprehensible, descriptions of the accompanying drawings are provided as follows.

FIG. 1A illustrates an analysis of harmonic centrality and betweenness centrality performed on diabetes sample data according to a method of an embodiment of the present invention.

FIG. 1B illustrates a comorbidity prediction model for diabetes constructed according to a method of an embodiment of the present invention.

FIGS. 1C to 1F illustrate disease association diagrams displayed when different disease codes are selected in the diabetes comorbidity prediction model.

FIG. 2A illustrates an analysis of harmonic centrality and betweenness centrality performed on conjunctival disease sample data according to a method of another embodiment of the present invention.

FIG. 2B illustrates a comorbidity prediction model for conjunctival diseases constructed according to the method of another embodiment of the present invention.

FIGS. 2C to 2F illustrate disease association diagrams displayed when different disease codes are selected in the conjunctival disease comorbidity prediction model.

FIG. 3 is a schematic diagram of the multi-layer network structure in one embodiment of the present invention.

### DETAILED DESCRIPTION

To provide a more comprehensive description of the implementation of the present invention, the following explanatory descriptions are provided for different aspects and specific embodiments. These are not limited to a particular form of implementation or application but encompass the features and method steps of multiple specific embodiments. Different embodiments can achieve the same or similar functions and steps, demonstrating the flexibility of the present invention.

The present invention provides a method for constructing a comorbidity prediction model of diseases. The method comprises (1) receiving a sample dataset that comprises a plurality of disease names or codes for different diseases and a plurality of patient counts for each respective disease; (2) conducting a pairwise chi-square test on each individual disease in the sample data to determine an association between each pair of diseases, and retaining disease relationships with a p-value less than a specified threshold; and (3) identifying at least one key core disease and at least one bridge disease by calculating harmonic centrality and betweenness centrality to establish the comorbidity prediction model.

The following describes an embodiment in which sample data from a database established using the ICD9CM disease code data of outpatient and inpatient patients from the Taiwan Landseed International Hospital from 2007 to 2015 was analyzed. The sample data comprises a total of 4,426,698 patient visits (male: 2,087,955 visits; female: 2,335,418 visits; the remainder with unknown gender), and 517,781 patients (male: 249,793 patients; female: 267,982 patients; the remainder with unknown gender). The sample data was then preprocessed as follows.
(a) Non-disease ICD9 codes, such as codes 780-799 (symptoms, signs, and ill-defined conditions), 800-999 (injuries and poisonings, E and V codes: external causes and supplementary classifications) are excluded.
(b) A predetermined threshold is applied to exclude data from the above-mentioned (a). The predetermined threshold includes cases where a single disease was consulted fewer than two times within a year (i.e., identifying cases where the same disease code (ICD9) was recorded in outpatient visits less than twice in one year), resulting in 517,464 remaining patients.
(c) A pairwise Chi-square test is conducted on the individual diseases in the sample data to determine the association between each pair of diseases, and disease relationships with a p-value less than a specified threshold are retained. Preferably, the specified threshold is 0.05, meaning that if the p-value of the Chi-square test between two diseases is less than 0.05, they are considered to have a disease relationship. For example, Table 1 below shows the result of the Chi-square test between diabetes (ICD9: 250) and hypertension (ICD9: 401). If the Chi-square test indicates a relationship between two diseases, a line is drawn to connect them. In total, 649 diseases and 95,786 disease relationships were identified through this process.

TABLE 1
Determination of the Association Between Diabetes
and Hypertension Using Chi-Square Test

| | | Diabetes | | |
|---|---|---|---|---|
| | | 1 | 0 | total |
| Hypertension | 1 | 14,474 | 12,937 | 27,411 |
| | 0 | 32,997 | 457,056 | 490,053 |
| | total | 47,471 | 469,993 | 517,464 |

P-value < 0.05

Next, the number of visits in the sample data was divided by quartiles to exclude diseases with lower visit counts. The top 75% of diseases with higher visit counts were retained to avoid statistical errors caused by diseases with fewer visits. After filtering, the number of diseases was 649, with 70,039 association links remaining.

To identify the core combinations within the network of significantly related diseases, harmonic centrality and betweenness centrality were calculated for the associated disease network data. This process identified a key core disease and a bridge disease, thereby establishing a comorbidity prediction model, as detailed below.

Harmonic centrality is used to identify key core diseases, with the calculation formula as follows:

$$H ( s ) = \frac{1}{n - 1} \cdot {\sum }_{s \neq t} \frac{1}{d ( s , t )}$$

where H(s) represents the harmonic centrality score; n is the number of disease nodes; s and t denote distinct disease nodes; and d(s,t) is the shortest path length from s to t. The threshold for harmonic centrality scores must be greater than the third quartile (Q3) of the network, thereby retaining the top 25% central nodes in the network.

Betweenness centrality is used to identify bridge diseases, with the calculation formula as follows:

$${C}_{B} ( v ) = {\sum }_{s \neq v \neq t} \frac{{σ}_{st} ( v )}{{σ}_{st}}$$

where CB(v) represents the betweenness centrality score; s, t, and v denote distinct disease nodes; σₛₜ(v) is a number of shortest paths from s to t that pass-through v; and σₛₜ is a total number of shortest paths from s to t. The threshold for betweenness centrality scores requires that the betweenness centrality must exceed 1.5 times the interquartile range (IQR).

Through the calculation of harmonic centrality and betweenness centrality, a total of 78 key core and bridge diseases were identified, as shown in Table 2 below. The threshold for harmonic centrality was set at a score>0.8. If a disease's harmonic centrality score exceeded this threshold, it was considered a core disease. The threshold for betweenness centrality was set at a score>590. If a disease's betweenness centrality score exceeded this threshold, it was considered a bridge disease. If both harmonic and betweenness centrality scores of a disease exceeded these thresholds, it was regarded as both a core and bridge disease.

TABLE 2
ICD9 Codes, Disease Names, Harmonic Centrality, and Betweenness
Centrality of the 78 Key Core and Bridge Diseases

| | | harmonic | betweenness |
|---|---|---|---|
| ICD9 | diseases | centrality | centrality |
| 038 | septicemia | 0.787 | 880.114 |
| 250 | diabetes mellitus | 0.838 | 707.943 |
| 285 | anemia | 0.829 | 768.693 |
| 372 | conjunctivitis | 0.823 | 596.044 |
| 401 | essential hypertension | 0.859 | 994.920 |
| 436 | acute, but ill-defined, cerebrovascular | 0.827 | 675.471 |
| | disease | | |
| 460 | acute nasopharyngitis | 0.907 | 1526.930 |
| 461 | acute sinusitis | 0.852 | 777.922 |
| 462 | acute pharyngitis | 0.845 | 833.878 |
| 463 | acute tonsillitis | 0.827 | 603.536 |
| 464 | acute laryngitis and acute tracheitis | 0.833 | 669.410 |
| 465 | acute upper respiratory infections | 0.920 | 1725.468 |
| 466 | acute bronchitis | 0.906 | 1634.768 |
| 470 | deviated nasal septum | 0.818 | 591.527 |
| 472 | chronic rhinitis and chronic pharyngitis | 0.883 | 866.602 |
| 477 | allergic rhinitis | 0.897 | 1154.307 |
| 478 | diseases of upper respiratory tract | 0.860 | 919.010 |
| 482 | bacterial pneumonia | 0.829 | 985.934 |
| 485 | bronchopneumonia, organism | 0.877 | 1238.641 |
| | unspecified | | |
| 486 | pneumonia, organism unspecified | 0.907 | 1993.737 |
| 487 | influenza | 0.823 | 740.139 |
| 490 | bronchitis | 0.858 | 894.194 |
| 491 | chronic bronchitis | 0.881 | 1434.213 |
| 493 | asthma | 0.887 | 1352.150 |
| 496 | chronic airways obstruction, | 0.904 | 1736.420 |
| | not elsewhere classified | | |
| 511 | pleurisy | 0.862 | 1004.377 |
| 518 | diseases of lung | 0.842 | 1517.957 |
| 521 | disease of hard tissues of teeth | 0.840 | 825.264 |
| 523 | gingival and periodontal disease | 0.840 | 716.920 |
| 525 | disorder of the teeth and supporting | 0.809 | 668.030 |
| | structures | | |
| 528 | diseases of the oral soft tissues | 0.854 | 1077.049 |
| 530 | disorder of esophagus | 0.884 | 1235.819 |
| 531 | gastric ulcer | 0.885 | 1290.517 |
| 532 | duodenal ulcer | 0.829 | 862.430 |
| 533 | peptic ulcer | 0.883 | 1336.463 |
| 535 | gastritis and gastroduodenitis | 0.863 | 1333.644 |
| 536 | disorders of function of stomach | 0.906 | 1212.219 |
| 558 | non-infectious gastroenteritis and colitis | 0.846 | 1022.164 |
| 560 | intestinal obstruction | 0.837 | 985.401 |
| 564 | functional gastrointestinal disorders | 0.940 | 1984.645 |
| 569 | disorder of intestine | 0.835 | 709.198 |
| 571 | chronic hepatitis and cirrhosis of liver | 0.900 | 1388.545 |
| 573 | disorder of liver | 0.887 | 1113.588 |
| 574 | calculus of bile duct | 0.872 | 740.699 |
| 577 | disease of pancreas | 0.854 | 1115.287 |
| 578 | hemorrhage of gastrointestinal tract | 0.908 | 1554.584 |
| 584 | acute renal failure | 0.803 | 620.305 |
| 585 | chronic renal failure | 0.845 | 1065.886 |
| 586 | renal failure, unspecified | 0.796 | 753.505 |
| 593 | disorders of kidney and ureter | 0.874 | 1176.290 |
| 596 | disorders of bladder | 0.843 | 709.475 |
| 599 | urinary tract and urethra disorders | 0.922 | 1535.641 |
| 600 | hypertrophy of prostate | 0.900 | 1223.672 |
| 611 | breast disorder | 0.795 | 721.713 |
| 614 | disease of female pelvic organs and | 0.845 | 709.661 |
| | tissues | | |
| 616 | diseases of cervix, vagina, and vulva | 0.815 | 735.749 |
| 626 | disorders of menstruation and other | 0.843 | 859.358 |
| | abnormal bleeding from female genital | | |
| | tract | | |
| 627 | menopausal and postmenopausal | 0.819 | 621.112 |
| | disorder | | |
| 680 | carbuncle and furuncle | 0.854 | 856.519 |
| 681 | cellulitis and abscess of fingers and toes | 0.836 | 624.170 |
| 682 | other cellulitis and abscess | 0.907 | 2225.105 |
| 692 | dermatitis and other eczema | 0.916 | 1581.261 |
| 698 | pruritic disorder | 0.846 | 740.319 |
| 707 | chronic ulcer of skin | 0.838 | 941.931 |
| 708 | urticaria | 0.796 | 876.032 |
| 709 | disorder of skin and subcutaneous tissue | 0.790 | 638.922 |
| 715 | osteoarthrosis, generalized or localized | 0.898 | 1674.136 |
| 716 | arthropathy | 0.845 | 739.469 |
| 719 | disorder of joint | 0.821 | 1010.945 |
| 721 | allied disorders of spine | 0.908 | 1315.348 |
| 722 | disc disorder | 0.837 | 733.658 |
| 724 | back disorders | 0.882 | 1124.303 |
| 726 | enthesopathy of ankle and tarsus | 0.870 | 925.896 |
| 727 | disorders of synovium, tendon, and | 0.863 | 857.922 |
| | bursa | | |
| 728 | disorders of muscle, ligament, and | 0.831 | 749.217 |
| | fascia | | |
| 729 | disorders of soft tissue | 0.886 | 1397.987 |
| 733 | disorders of bone and cartilage | 0.886 | 989.669 |
| 756 | anomalies of musculoskeletal system | 0.802 | 597.267 |

As shown in Table 2, diseases with ICD9 codes 038, 586, 611, 708, and 709 have lower harmonic centrality values and are thus identified solely as bridge diseases, while the remaining diseases are identified as both core and bridge diseases. This forms a comprehensive comorbidity network for the hospital, illustrating the interrelationships among various diseases. These findings can serve as a predictive tool, estimating the likelihood that patients previously diagnosed with one of these diseases at the hospital may later be diagnosed with other related diseases. This information is valuable for regional preventive healthcare planning.

Next, each of the 78 diseases can be individually extended to establish 78 separate comorbidity prediction models for each specific disease.

Furthermore, this invention can be applied to single diseases for disease network analysis. An example is provided below for illustration.

### Example 1: Diabetes (ICD9: 250)

The sample data comes from the outpatient and inpatient records of diabetic patients at Landseed International Hospital in Taiwan between 2007 and 2015, including data for diabetic patients with comorbidities. A total of 391 associated diseases were identified, forming 38,144 disease networks. A Chi-square test was performed for each disease pair in the sample data to assess the association between every two diseases, and only the disease relationships with a P-value below a specific threshold, preferably 0.05, are retained. The number of visits for each disease in the sample data was divided into quartiles, retaining the diseases with the top 50% of visit counts. Next, association rules were applied to calculate the lift between each pair of diseases using the formula: P(B|A)/P(B), where A and B represent two distinct diseases. A higher lift value indicates a stronger association between the two diseases, while a lower lift suggests a negative correlation. Diseases in the top 25% of lift values were retained, resulting in 137 associated diseases and 635 disease relationships within the disease network.

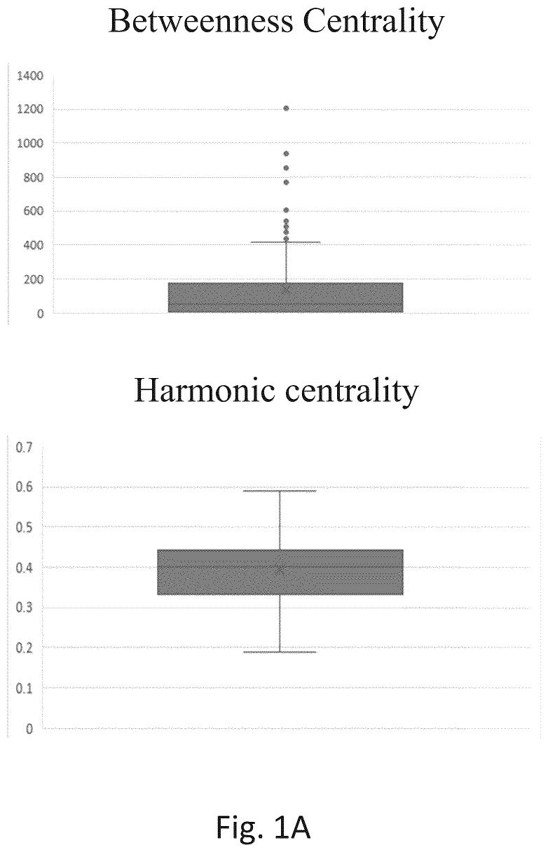

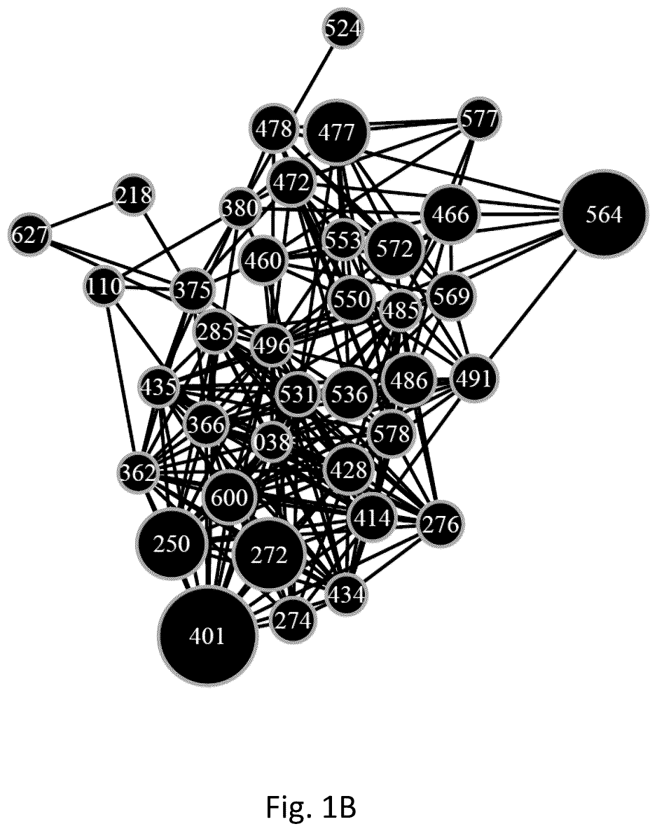

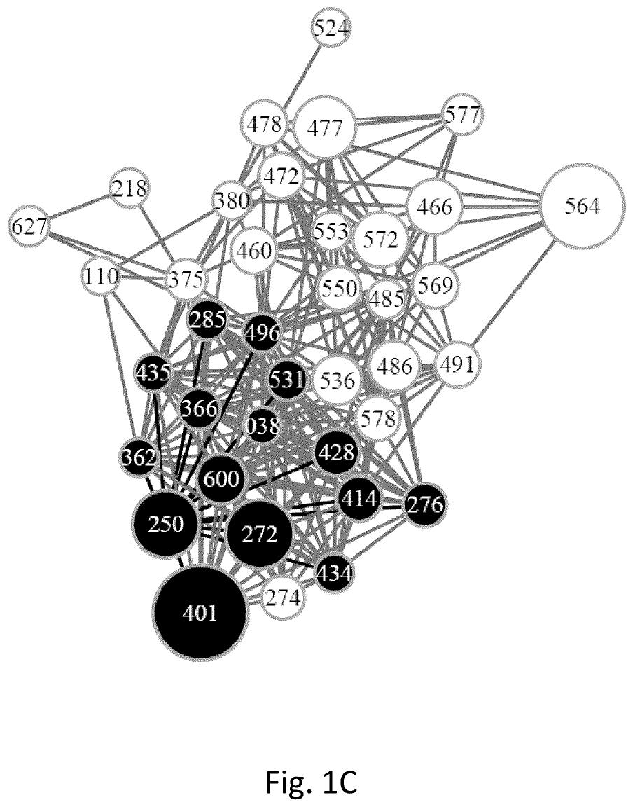

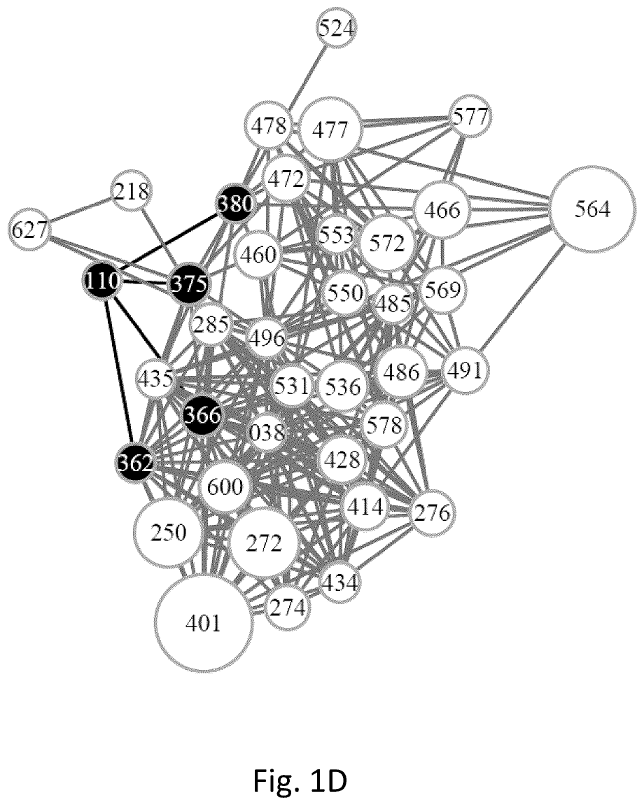

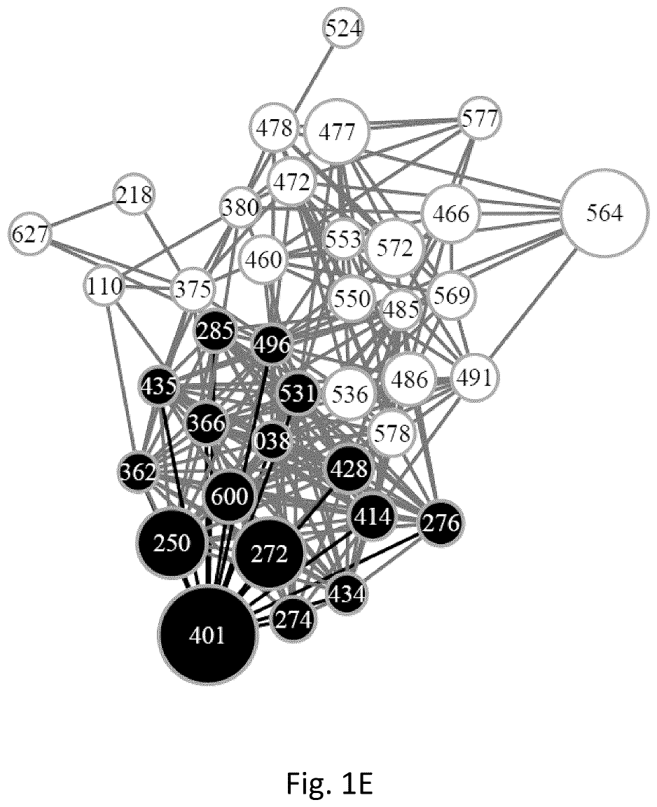

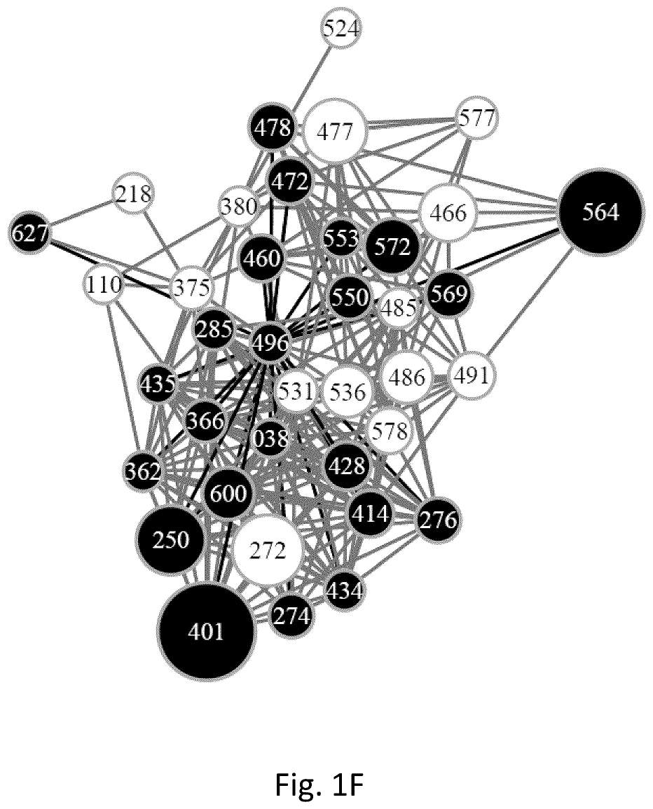

To identify the core combinations within the key diabetes disease network, harmonic centrality and betweenness centrality were calculated for the diabetes-related disease network data, as shown in FIG. 1A. The calculation formulas are as previously described and will not be repeated here. The results were filtered using quartiles, retaining disease combinations with harmonic centrality scores in the top 25% (scores >0.443) and betweenness centrality scores greater than 1.5 times the interquartile range (IQR) (scores >415). This analysis identified 38 key core and bridge diseases, forming a disease network with 227 disease relationships, as shown in FIG. 1B. The size of each circle represents the number of patients associated with the disease; the larger the circle, the higher the number of patients. The numbers in FIG. 1B correspond to the ICD9-CM disease codes, as listed in Table 3 below.

TABLE 3
ICD9 Codes, Disease Names, Harmonic Centrality,
and Betweenness Centrality for the 38 Key Core
and Bridge Diseases Associated with Diabetes.

| | | harmonic | betweenness |
|---|---|---|---|
| ICD9 | diseases | centrality | centrality |
| 038 | septicemia | 0.521 | 394.034 |
| 110 | dermatophytosis | 0.435 | 504.323 |
| 218 | leiomyoma of uterus | 0.418 | 497.170 |
| 250 | diabetes mellitus | 0.467 | 14.180 |
| 272 | disorders of lipoid metabolism | 0.449 | 33.886 |
| 274 | gout | 0.445 | 229.437 |
| 276 | electrolyte and fluid disorders | 0.486 | 146.896 |
| 285 | anemia | 0.508 | 415.926 |
| 362 | retinal disorders | 0.504 | 262.204 |
| 366 | cataract | 0.524 | 407.466 |
| 375 | disorders of lacrimal system | 0.493 | 459.616 |
| 380 | disorder of external ear | 0.491 | 940.003 |
| 401 | essential hypertension | 0.477 | 13.641 |
| 414 | chronic ischemic heart disease | 0.507 | 40.016 |
| 428 | heart failure | 0.515 | 111.904 |
| 434 | cerebral embolism, cerebral infarction, | 0.499 | 28.006 |
| | cerebral thrombosis | | |
| 435 | transient cerebral ischemias | 0.565 | 852.082 |
| 460 | acute nasopharyngitis | 0.507 | 435.952 |
| 466 | acute bronchitis | 0.445 | 76.924 |
| 472 | chronic rhinitis and chronic pharyngitis | 0.503 | 474.752 |
| 477 | allergic rhinitis | 0.475 | 157.394 |
| 478 | diseases of upper respiratory tract | 0.463 | 147.200 |
| 485 | bronchopneumonia, organism | 0.521 | 368.818 |
| | unspecified | | |
| 486 | pneumonia, organism unspecified | 0.483 | 85.541 |
| 491 | chronic bronchitis | 0.485 | 136.454 |
| 496 | chronic airways obstruction, | 0.588 | 1203.522 |
| | not elsewhere classified | | |
| 524 | dentofacial anomalies | 0.346 | 536.684 |
| 531 | gastric ulcer | 0.556 | 770.504 |
| 536 | disorders of function of stomach | 0.475 | 120.927 |
| 550 | hernia | 0.456 | 63.261 |
| 553 | disorder of intestine | 0.456 | 73.857 |
| 564 | functional gastrointestinal disorders | 0.469 | 161.103 |
| 569 | disorders of intestine | 0.459 | 64.092 |
| 572 | sequelae of chronic liver disease | 0.451 | 51.547 |
| 577 | disease of pancreas | 0.438 | 513.352 |
| 578 | hemorrhage of gastrointestinal tract | 0.528 | 606.453 |
| 600 | hypertrophy of prostate | 0.531 | 351.829 |
| 627 | menopausal and postmenopausal | 0.482 | 788.537 |
| | disorder | | |

As shown in Table 3, the ICD9 codes 496, 435, 531, 578, 285, 460, 472, 375, 380, and 627 are both core diseases and bridge diseases. The ICD9 codes 600, 366, 485, 038, 428, 414, 362, 434, 276, 491, 486, 401, 477, 536, 564, 250, 478, 569, 550, 553, 572, 272, 274, 466 are core diseases, while the ICD9 codes 524, 577, 110, and 218 are bridge diseases. This forms a diabetes comorbidity network, which includes the comorbidity relationships between different diseases, and integrates information on the number of patients and lift values. This network can help physicians or patients proactively engage in prevention or treatment. If a patient is diagnosed with one of the diseases in the network, the network's connections can predict other diseases the patient might develop in the future. Moreover, by calculating the proportion of patients with these diseases within the network, the probability of developing such diseases can be estimated. Using the lift values from association rules, the risk of disease can be further evaluated, offering more precise health risk assessments.

For example, if a patient has diabetes, the potential future diseases they may develop are 14 in total (ICD9 codes: 038, 272, 276, 285, 362, 366, 401, 414, 428, 434, 435, 496, 531, 600), as shown in FIG. 1C. If the patient has both diabetes and dermatophytosis (ICD9 code: 110, a bridge disease), then in addition to the 14 diseases listed above, two additional diseases (ICD9 codes: 375, 380) should also be noted, as shown in FIG. 1D. If the patient has diabetes and hypertension (ICD9 code: 401, a core disease), then in addition to the 14 diseases, one more disease (ICD9 code: 274) should be noted, as shown in FIG. 1E. If the patient has diabetes and chronic airways obstruction disease (496, which is both a core and bridge disease), then 10 additional diseases (ICD9 codes: 274, 460, 472, 478, 550, 553, 564, 569, 572, 627) should be noted, as shown in FIG. 1F.

### Example 2: Conjunctival Disorders (ICD9CM: 372)

The sample data comes from outpatient and inpatient records of patients with conjunctival disorders from Landseed International Hospital in Taiwan, covering the period from 2007 to 2015. This dataset includes cases where patients had conjunctival disorders along with other conditions, totaling 383 related diseases and forming 37,862 interconnected disease networks. A chi-square test was conducted on individual diseases in the sample to determine the association between each pair of diseases, and disease relationships with a p-value below a specific threshold were retained; a preferred threshold is 0.05. The sample data's visit counts were divided into quartiles, with the top 50% of diseases in terms of visit counts retained. Next, an association rule algorithm was applied to calculate the lift between each pair of diseases, with the formula P(B|A)/P(B), where A and B represent two distinct diseases. A higher lift indicates a stronger association, while a lower lift signifies a negative correlation. The top 25% of diseases based on lift were retained, resulting in a disease network comprising 128 related diseases and 599 disease relationships.

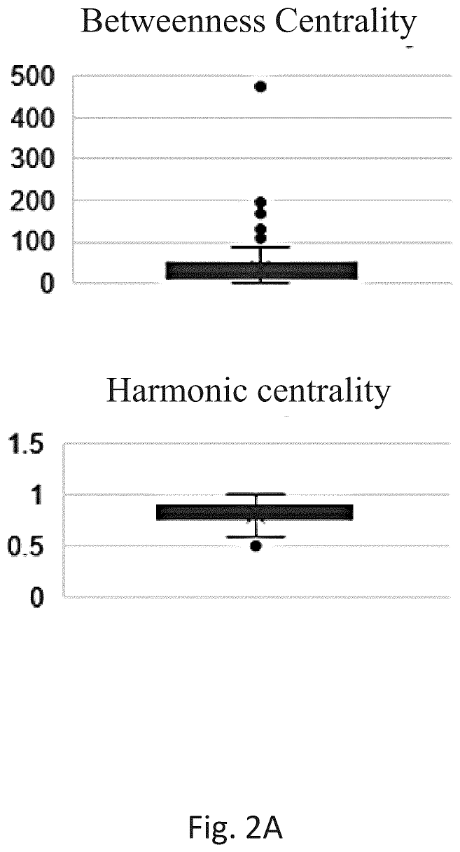

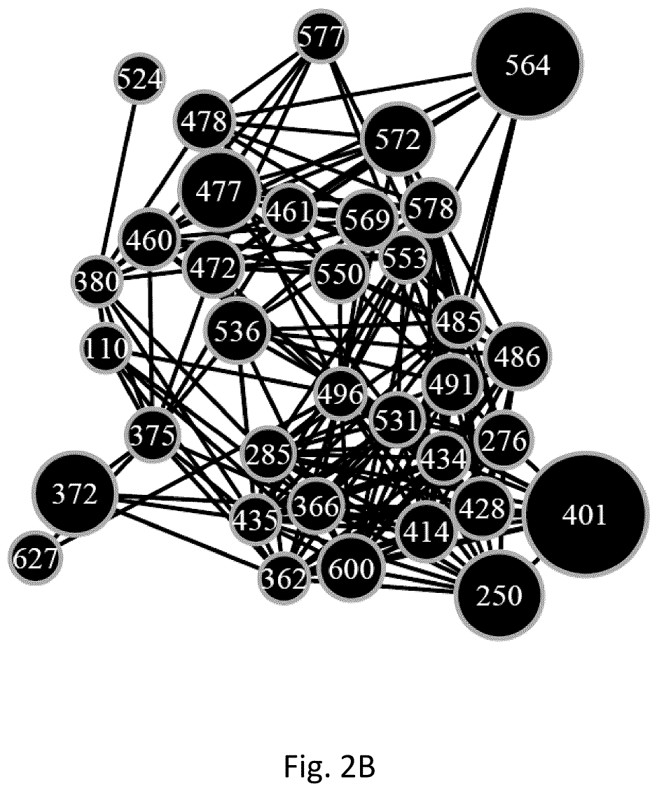

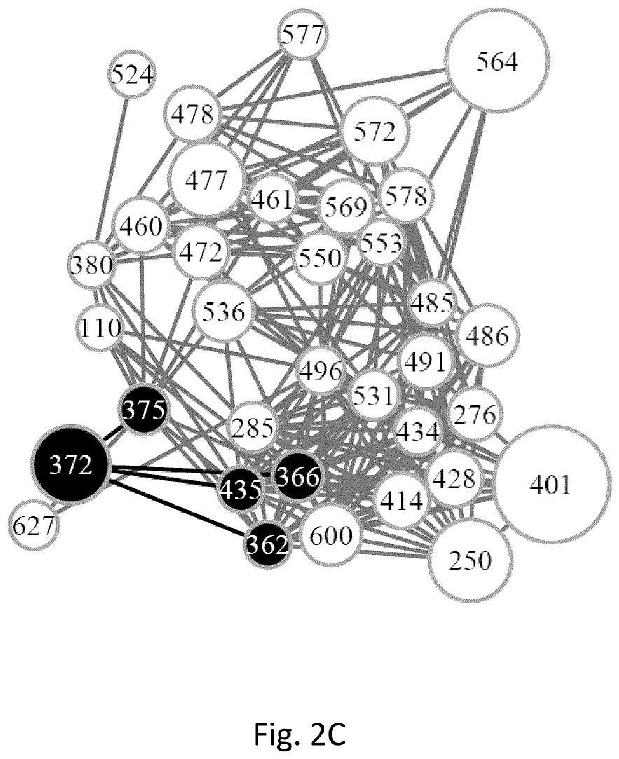

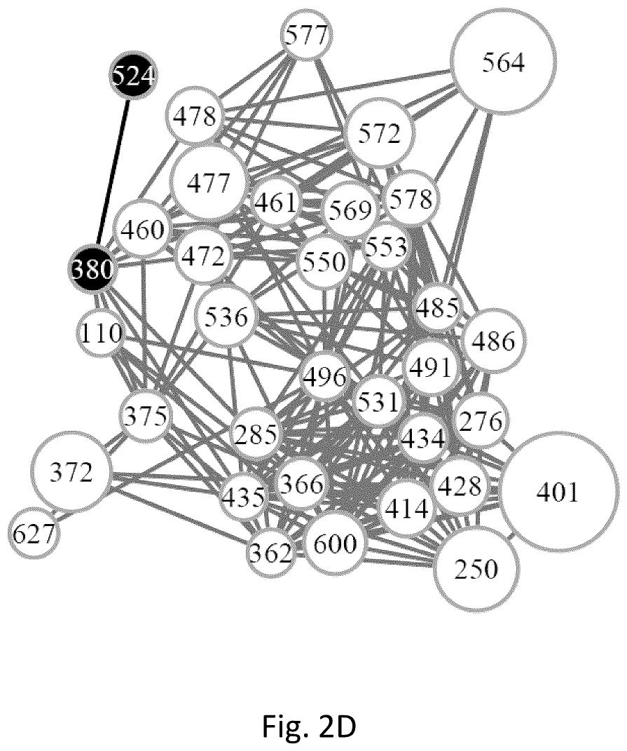

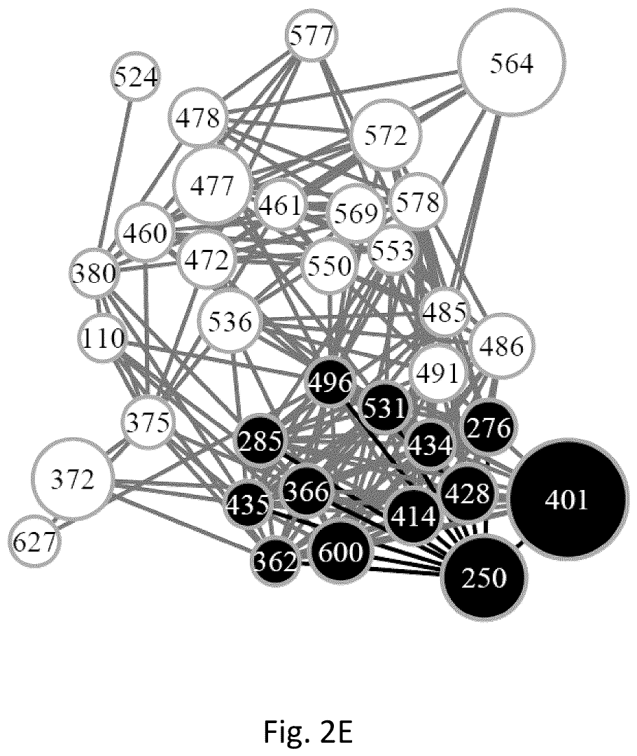

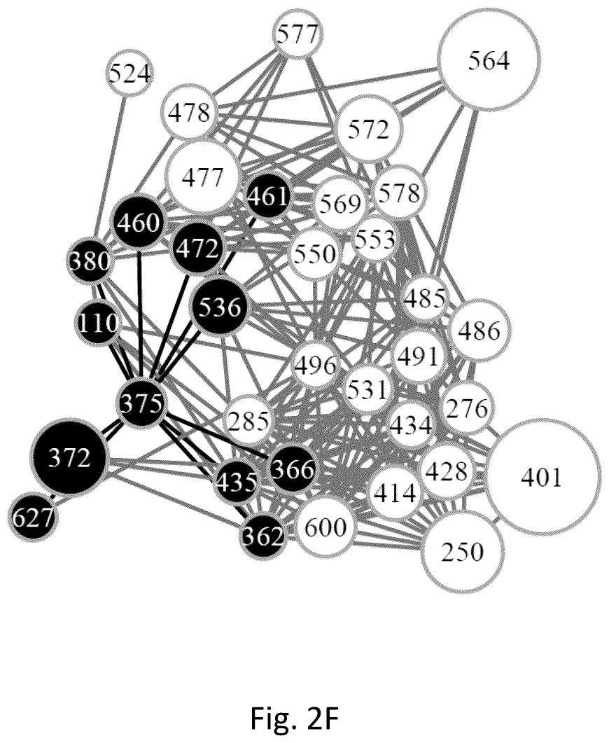

The conjunctival disorder-related disease network data underwent calculations for harmonic centrality and betweenness centrality, as shown in FIG. 2A, with the calculation formulas previously explained and not repeated here. Quartile filtering was applied to the results to select disease combinations with harmonic centrality scores in the top 25% (score>0.45) and betweenness centrality scores exceeding 1.5 IQR (score>400). This process identified a disease network with 35 key core and bridging diseases, forming 198 disease connections, as illustrated in FIG. 2B. In FIG. 2B, the size of each circle indicates the number of patients with that disease, with larger circles representing higher patient counts. The numbers shown in FIG. 2B correspond to ICD9 codes for these diseases, detailed in Table 4 below.

TABLE 4
Key Core and Bridging Diseases Associated with Conjunctival
Disorders - ICD9 Codes, Disease Names, Harmonic
Centrality, and Betweenness Centrality.

| | | harmonic | betweenness |
|---|---|---|---|
| ICD9 | diseases | centrality | centrality |
| 110 | dermatophytosis | 0.477 | 493.373 |
| 250 | diabetes mellitus | 0.465 | 10.891 |
| 276 | electrolyte and fluid disorders | 0.489 | 138.861 |
| 285 | anemia | 0.508 | 348.258 |
| 362 | retinal disorders | 0.507 | 166.832 |
| 366 | cataract | 0.528 | 260.316 |
| 372 | conjunctivitis | 0.402 | 4.891 |
| 375 | disorders of lacrimal system | 0.518 | 520.869 |
| 380 | disorder of external ear | 0.502 | 796.241 |
| 401 | essential hypertension | 0.476 | 12.680 |
| 414 | chronic ischemic heart disease | 0.512 | 66.183 |
| 428 | heart failure | 0.516 | 111.201 |
| 434 | cerebral embolism, cerebral infarction, | 0.499 | 29.644 |
| | cerebral thrombosis | | |
| 435 | transient cerebral ischemias | 0.574 | 714.219 |
| 460 | acute nasopharyngitis | 0.509 | 318.098 |
| 461 | acute sinusitis | 0.464 | 89.663 |
| 472 | chronic rhinitis and chronic pharyngitis | 0.499 | 270.851 |
| 477 | allergic rhinitis | 0.482 | 130.940 |
| 478 | diseases of upper respiratory tract | 0.468 | 137.156 |
| 485 | bronchopneumonia, organism | 0.509 | 236.110 |
| | unspecified | | |
| 486 | pneumonia, organism unspecified | 0.472 | 51.900 |
| 491 | chronic bronchitis | 0.491 | 181.118 |
| 496 | chronic airways obstruction, not | 0.590 | 1057.239 |
| | elsewhere classified | | |
| 524 | dentofacial anomalies | 0.352 | 494.655 |
| 531 | gastric ulcer | 0.555 | 703.781 |
| 536 | disorders of function of stomach | 0.507 | 208.772 |
| 550 | hernia | 0.462 | 60.805 |
| 553 | disorder of intestine | 0.461 | 72.872 |
| 564 | functional gastrointestinal disorders | 0.467 | 136.093 |
| 569 | disorder of intestine | 0.466 | 62.855 |
| 572 | sequelae of chronic liver disease | 0.462 | 57.717 |
| 577 | disease of pancreas | 0.447 | 480.930 |
| 578 | hemorrhage of gastrointestinal tract | 0.522 | 480.560 |
| 600 | hypertrophy of prostate | 0.542 | 391.411 |
| 627 | menopausal and postmenopausal | 0.482 | 572.708 |
| | disorder | | |

As shown in Table 4, the following ICD9 codes represent diseases that are both core and bridging diseases: 496, 380, 435, 531, 627, 375, 110, and 578. Additionally, the following ICD9 codes represent core diseases: 600, 366, 428, 414, 485, 460, 285, 536, 362, 472, 434, 491, 276, 477, 401, 486, 478, 564, 569, 250, 461, 572, 550, and 553. Codes 524 and 577 represent bridging diseases. By establishing a conjunctival disease comorbidity network that incorporates the relationships between various diseases, as well as patient incidence and lift values, this network can help doctors and patients proactively pursue preventive measures or treatments. Through this network, future risks of associated diseases can be predicted based on the presence of conjunctival disorders, enhancing regional preventive healthcare strategies.

For example, if a patient has a conjunctival disease, they may be at risk of developing four additional diseases in the future, identified by ICD9 codes: 362, 366, 375, and 435, as shown in FIG. 2C. If the patient has both a conjunctival disease and dentofacial anomalies (ICD9 code: 524, a bridging disease), they should also be aware of an additional disease (ICD9 code: 380), as depicted in FIG. 2D. If the patient has a conjunctival disease along with diabetes (ICD9 code: 250, a core disease), there are nine additional diseases they may need to monitor, identified by ICD9 codes: 276, 285, 401, 414, 428, 434, 496, 531, and 600, as shown in FIG. 2E. For a patient with both a conjunctival disease and a lacrimal system disease (ICD9 code: 375, which is both a core and bridging disease), seven more diseases may need attention, identified by ICD9 codes: 110, 380, 460, 461, 472, 536, and 627, as illustrated in FIG. 2F.

Additionally, because complex diseases influence each other in ways that go beyond a one-to-one relationship, a hospital-wide dataset can be used to calculate the average network length. This allows for the construction of a multi-layer network structure. The calculation formula is as follows:

$${l}_{g} = \frac{1}{E \cdot ( E - 1 )} \cdot {\sum }_{s \neq t} d ( s , t )$$

where lg represents the network average path length; E is the number of connections between any two of the disease nodes; s and t denote distinct disease nodes; and d(s,t) is the shortest path length from s to t. The network average path length determines the number of layers in the multi-layer network structure.

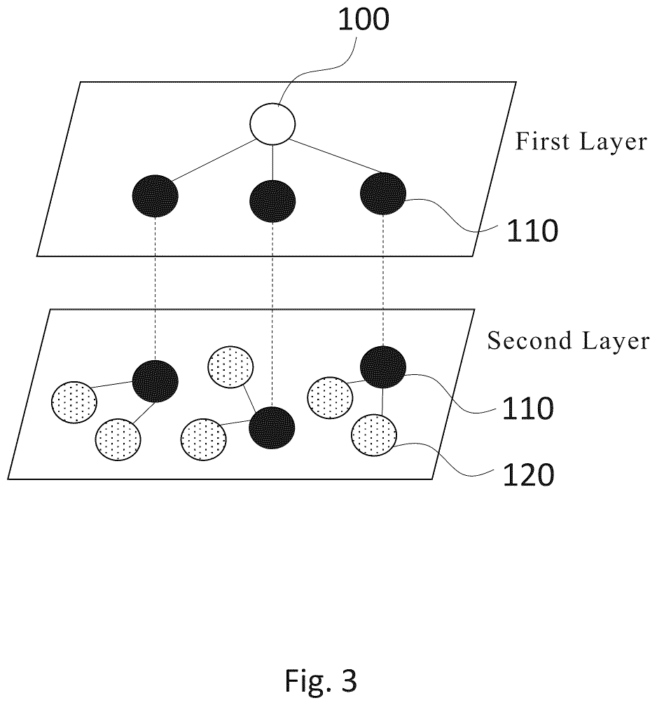

Using sample data on ICD9 codes from outpatient and inpatient records at Landseed International Hospital in Taiwan from 2007-2015, a comprehensive hospital disease network was constructed, encompassing 649 diseases and 70,039 connections. Analysis of this network showed an average path length of 2, as illustrated in FIG. 3. This allows for the construction of a two-layer network structure. The first layer includes a specified disease 100 under analysis (e.g., diabetes) and associated comorbid diseases 110 (e.g., hypertension, retinal disease, chronic airway obstruction). The second layer includes these comorbid diseases 110 and secondary comorbid diseases 120 (e.g., hypertensive heart disease, myocardial infarction, heart failure linked to hypertension, and glaucoma, cataracts linked to retinal disease) related to each of the comorbid diseases 110. Further layers can be built in this manner as required. It is important to note that the number of layers in this multi-layer network and the number of the comorbid diseases included at each layer will vary based on the contents of the sample data from the received database. Thus, even network structures focused on diabetes may present different layers or disease relationships depending on the specifics of the different sample data.

In summary, the method provided by this invention for constructing a comorbidity prediction model of diseases involves preprocessing the received sample data and then applying harmonic centrality and betweenness centrality analyses. This approach identifies key core diseases (those most centrally connected to all others, with equidistant access to all nodes) and bridge diseases (those serving as connectors between different disease categories) within the network, forming the basis of the comorbidity prediction model. By leveraging this comorbidity prediction model, one can explore comorbidity relationships between a specific disease and other diseases. Through the proportion of comorbid cases and lift values, it is possible to estimate comorbidity risks, offering valuable insights for early disease prevention.

While the embodiments of the present invention have been disclosed as above, they are not intended to limit the invention. Those skilled in the art may make various modifications and refinements without departing from the spirit and scope of the invention. Therefore, the scope of protection for this invention shall be defined by the appended claims.

What is claimed is:

1. A method for constructing a comorbidity prediction model of diseases, comprising the following steps:
(1) receiving a sample dataset that comprises a plurality of disease names or codes for different diseases and a plurality of patient counts for each respective disease;
(2) conducting a pairwise chi-square test on each individual disease in the sample data to determine an association between each pair of diseases, and retaining disease relationships with a p-value less than a specified threshold; and
(3) identifying at least one key core disease and at least one bridge disease by calculating harmonic centrality and betweenness centrality to establish the comorbidity prediction model.

2. The method of claim 1, further comprising excluding data from the sample dataset based on a predetermined threshold, wherein the predetermined threshold comprises diseases with fewer than two consultations within a one-year period before performing step (1).

3. The method of claim 1, further comprising categorizing the patient counts in the sample dataset by quartiles and retaining diseases with patient counts in the top 50% or 75% after performing step (2).

4. The method of claim 1, further comprising, after performing step (2):
calculating a lift between each pair of the diseases in the sample dataset using an association rule and selecting; and
retaining diseases with lift values in the top 25%.

5. The method of claim 1, wherein the specified threshold is 0.05.

6. The method of claim 1, wherein
the harmonic centrality is calculated by a formula of
$$H ( s ) = \frac{1}{n - 1} \cdot {\sum }_{s \neq t} \frac{1}{d ( s , t )} ,$$
where H(s) represents the harmonic centrality score,
n is the number of disease nodes,
s and t denote distinct disease nodes, and
d(s,t) is the shortest path length from s to t; and
the betweenness centrality is calculated by a formula of
$${C}_{B} ( v ) = {\sum }_{s \neq v \neq t} \frac{{σ}_{st} ( v )}{{σ}_{st}} ,$$
where CB(v) represents the betweenness centrality score,
s, t, and v denote distinct disease nodes,
σₛₜ(v) is a number of shortest paths from s to t that pass-through v, and
σₛₜ is a total number of shortest paths from s to t.

7. The method of claim 6, wherein nodes with harmonic centrality scores in the top 25% are retained.

8. The method of claim 6, wherein the threshold for the betweenness centrality is greater than 1.5 times the interquartile range (IQR).

9. The method of claim 1, further comprising:
calculating the network average path length of the sample dataset to construct a multi-layer network structure by a formula of
$${l}_{g} = \frac{1}{E \cdot ( E - 1 )} \cdot {\sum }_{s \neq t} d ( s , t ) ;$$
where lg represents the network average path length,
E is the number of connections between any two of the disease nodes,
s and t denote distinct disease nodes, and
d(s,t) is the shortest path length from s to t;
wherein the network average path length determines the number of layers in the multi-layer network structure.
